(19) United States
(12) Patent Application Publication
(10) Pub. No.: US 2026/0152460 A1
(43) Pub. Date: Jun. 4, 2026

(54) PROCESS TO MINIMIZE POLYPHOSPHINE USAGE BY MAKING USE OF DEGRADATION PRODUCTS

(71) Applicant: Dow Technology Investments LLC, Midland, MI (US)

(72) Inventors: Michael A. Brammer, Lake Jackson, TX (US); William N. Felsted, II, Lake Jackson, TX (US); Yuko Kida, Zionsville, IN (US); Amarnath Singh, Pearland, TX (US); Christophe R. Laroche, Lake Jackson, TX (US)

(21) Appl. No.: 19/125,333

(22) PCT Filed: Nov. 15, 2023
(86) PCT No.: PCT/US2023/079795

§ 371 Date: Apr. 29, 2025

Related U.S. Application Data
(60) Provisional application No. 63/432,078, filed on Dec. 13, 2022.

(51) Int. Cl. C07C 45/50

(52) U.S. Cl. CPC C07C 45/505

(57) ABSTRACT

The present invention relates in part to a method for slowing the polyphosphine ligand usage rate in a hydroformylation process. It has been discovered that when a target concentration of total polyphosphorous compounds comprising polyphosphine ligand and polydentate ligand degradation products is maintained in a reaction zone, the usage rate of the polyphosphine ligand may be reduced.

### FIELD

The present invention relates to a catalyst composition and methods of reducing the polyphosphine ligand usage in a hydroformylation process using that composition.

### BACKGROUND

It is known in the art that aldehydes may be readily produced by reacting an olefinically unsaturated compound with carbon monoxide and hydrogen in the presence of a solubilized rhodium-triorganophosphorous ligand complex catalyst and that a preferred type of such processes involves continuous hydroformylation. For example, U.S. Pat. No. 3,527,809 discloses the hydroformylation of alpha-olefins to produce aldehydes at low temperatures and pressures. The process employs certain rhodium complexes to effectively catalyze, under a defined set of variables in the presence of select triorganophosphorous ligands, the hydroformylation of olefins with hydrogen and carbon monoxide.

Among the catalysts described in U.S. Pat. No. 3,527,809 are compounds containing rhodium and triarylphosphorous ligands, in particular triarylphosphine ligands exemplified by triphenylphosphine (“TPP”). Commercial hydroformylation processes have successfully employed the rhodium-TPP catalyst for decades, and a key aspect of operation is the use of a large excess of TPP relative to rhodium. For example, industrial propylene hydroformylation processes often operate with TPP concentrations of 10-12 weight percent based on the total mass of the reaction fluid. Such high concentrations of TPP are used to achieve the desired product regioselectivity and to enhance catalyst stability.

Over time, hydroformylation catalysts tend to deactivate due to a plurality of causes including rhodium clustering or the presence of inhibitory compounds. Steps may be taken to maintain production targets, such as increasing the rhodium concentration, raising reaction temperatures, or catalyst rejuvenation treatments such as the procedure taught in U.S. Pat. No. 5,237,106. Despite these efforts, catalysts eventually reach a point where deactivation has rendered them nonviable for commercial use. These “end of life” catalysts must be removed from the process, sent off for precious metal recovery (PMR), and replaced with fresh charges of rhodium and ligand(s). The costs associated with PMR, replacing the entire rhodium and ligand inventories and the loss of production during the catalyst change can be significant.

Hydroformylation processes employing C3 and higher olefins produce a mixture of linear (normal) and branched (iso) aldehyde; the regioselectivity is typically expressed as the normal to isoaldehyde ratio (N/I). While both products are useful, the linear isomer often has more value in the marketplace; thus, it is generally desirable to produce a higher N/I ratio. It is well known that catalysts capable of producing an N/I in excess of about 10-12 are comprised of chelating ligands containing at least two phosphorous moieties (see for example “Rhodium Catalyzed Hydroformylation, Kluwer Academic Publishers, 2000). In general, chelating ligands include polyphosphines, polyphosphites, polyphosphoramidites and the like. Specific examples include but are not limited to triphosphines such as described in WO2018103536 and tetraphosphine ligands such as those described in U.S. Pat. Nos. 7,531,698 and 9,687,837.

Such chelating ligands are typically prepared via custom syntheses which increases their cost. In general, the desired amount of chelating ligand in the catalyst solution is greater than one molar equivalent relative to catalytic metal to ensure good performance (such as N/I values above 10) and no loss of catalytic metal; a typical range is from 1.2 to 5 moles of chelating ligand to metal and the optimal ratio to achieve a balance between performance and cost is typically around 3 moles chelating ligand to metal.

Chelating ligands are known to degrade during continuous operation due to oxidation, hydrolysis, or rhodium-promoted side reactions such as aryl cleavage. Much of the prior art teaches means to remove these degradation products since many are catalyst poisons or inhibitors (U.S. Pat. Nos. 4,605,780, 4,861,918, and 5,364,950) or contribute to autocatalytic ligand degradation. U.S. Pat. No. 4,260,828 teaches that one such inhibitory ligand degradation product (alkyl-diaryl phosphines, R—P(Ar)₂) can be used to mitigate further activity decline, but the inhibited catalyst exhibits a lower hydroformylation rate; thus, more drastic operating conditions must be used which tend to generate more heavy byproducts.

Because rhodium accountability, hydroformylation rate, and/or product regioselectivity will suffer if the chelating ligand concentration falls below one molar equivalent relative to rhodium, it is common practice to replenish these compounds to maintain the desired performance. The rate at which the chelating ligand must be added to the system to maintain the target concentration (“ligand usage rate”) is a key economic factor, since the chelating ligand is typically an expensive, custom-manufactured compound. For example, in a continuous hydroformylation process comprising a chelating polyphosphine, the goal would be to maintain the polyphosphine:rhodium ratio at an optimized target of roughly 3:1. Maintaining this polyphosphine:rhodium ratio involves the cost of continuously adding the expensive polyphosphine ligand to compensate for polyphosphine degradation.

U.S. Ser. No. 11/130,725 teaches adding monophosphines to catalysts comprising rhodium and tetraphosphine ligands to enhance catalyst stability. U.S. Ser. No. 11/344,869 also teaches that the ratio of the tetraphosphine and/or monophophines relative to the catalytic metal can be used to deliberately control the N/I.

A tetradentate substituted phosphane derivative was shown to be able to continue hydroformylation despite considerable ligand degradation in batch-mode operation but reaction rate and N/I performance data was not provided (Fanding Zhou, Lin Zhang, Qianhui Wu, Fuya Guo, Songbai Tang, Bin Xu, Maolin Yuan, Haiyan Fu, Ruixiang Li, Xueli Zheng, Hua Chen; Appl Organometal Chem. 2019; 33:e4646). The ligand degradation was reported to occur in the batch distillation process thus there is no teaching on how to maintain stable performance in a continuous hydroformylation process.

It would be desirable to have a method for slowing or reducing the ligand usage rate for high N/I hydroformylation processes comprising polyphosphines.

### SUMMARY

The present invention relates to slowing the polyphosphine ligand usage rate in a hydroformylation process. It has surprisingly been discovered that when a target concentration of total polyphosphorous compounds comprising polyphosphine ligand and polydentate ligand degradation products is maintained in a reaction zone, the usage rate of the polyphosphine ligand may be reduced.

In one embodiment, a method for slowing the polyphosphine ligand usage rate in a hydroformylation process comprises:
(a) contacting an olefin with carbon monoxide, hydrogen and a catalyst, the catalyst comprising (A) a transition metal; (B) a polyphosphine having the following structure:

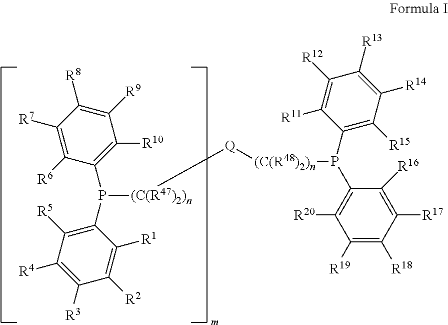

wherein each P is a phosphorous atom, and each of R¹-R²⁰ are independently hydrogen, a C1 to C8 alkyl group, a C1 to C8 alkoxyl group, an aryl group, an alkaryl group, or a halogen, R⁴⁷ and R⁴⁸ are independently hydrogen, a C1 to C8 alkyl group, or a C1 to C8 substituted alkyl group; n is 0 or 1, m is 2 or higher, and Q is a m+1 valent organic radical, wherein R⁵ and R⁶ and R¹⁵ and R¹⁶ may be links to form bridged structures, and, optionally, (C) a monophosphine having the following structure:

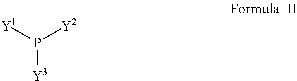

wherein P is a phosphorous atom, and each of Y¹-Y³ are independently an aryl group or a substituted aryl group, the contacting conducted in one or more reaction zones and at hydroformylation conditions; and
(b) monitoring the levels of total polyphosphorous compounds and
(c) adjusting the feed rate of the polyphosphine ligand to maintain the total polyphosphorous compounds concentration at a target value relative to the moles of transition metal.

The structure of the organic radical Q moiety is not narrowly important for the current invention other than the phosphorous moieties are positioned such that they can coordinate with the catalyst metal in a chelating fashion. Preferably, there should be no more than 10 bonds between the phosphorous atoms. Rigid groups such as naphthyl and biphenyl moieties may introduce additional restrictions related to the ability for chelation as is well known in the art. The preferred structure for Q is a tetrasubstituted biphenyl radical and most preferably with the phosphorous moieties at the 2, 2′, 6, 6′-isomer positions.

The organic radical Q is distinct from and not intended to be part of an insoluble polymeric resin since such a supported catalyst is incompatible with the present invention in that there is no continuous addition of a polymeric ligand as described in our process.

In some embodiments the preferred range of the values for m is 3 to 10, preferably less than 6. In some preferred embodiments, m is 3 or 4.

In one embodiment, a method for slowing the polyphosphine ligand usage rate in a hydroformylation process comprises the same as the previous embodiment except the monophosphine of Formula II is not used.

In one embodiment, a method for slowing the polyphosphine ligand usage in a hydroformylation process comprises:
(a) contacting an olefin with carbon monoxide, hydrogen and a catalyst, the catalyst comprising (A) a transition metal; (B) a tetraphosphine having the following structure:

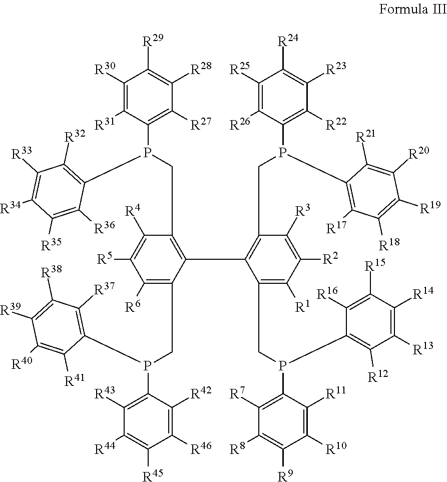

wherein each P is a phosphorous atom, and each of R¹-R⁴⁶ are independently hydrogen, a C1 to C8 alkyl group, an aryl group, an alkaryl group, or a halogen, and, optionally, (C) a monophosphine having the following structure:

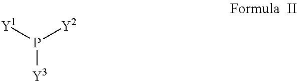

wherein P is a phosphorous atom, and each of Y¹-Y³ are independently an aryl group or a substituted aryl group, the contacting conducted in one or more reaction zones and at hydroformylation conditions; and
(b) monitoring the levels of total polyphosphorous compounds and
(c) adjusting the feed rate of the polyphosphine ligand to maintain the sum of the total polyphosphorous compounds to a set value relative to the moles of transition metal.

In one embodiment, a method for slowing the polyphosphine ligand usage in a hydroformylation process comprises:
(a) contacting an olefin with carbon monoxide, hydrogen and a catalyst, the catalyst comprising (A) a transition metal; (B) a tetraphosphine having the following structure:

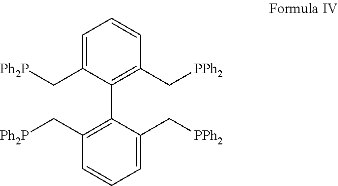

(b) adding triphenylphosphine to a reaction zone; and
(c) monitoring the levels of total polyphosphorous compounds and polyphosphine ligand and
(d) adjusting the feed rate of the polyphosphine ligand to maintain total polyphosphorous compounds to a set value relative to the moles of transition metal.

In one embodiment, a method for slowing the polyphosphine ligand usage in a hydroformylation process comprises:
a. contacting an olefin with carbon monoxide, hydrogen and a catalyst, the catalyst comprising (A) a transition metal; (B) triarylphosphine with the following structure:

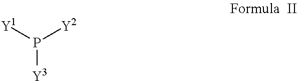

wherein P is a phosphorous atom, and each of Y¹-Y³ are independently an aryl group or a substituted aryl group, the contacting conducted in one or more reaction zones and at hydroformylation conditions
b. adding a tetraphosphine having the following structure:

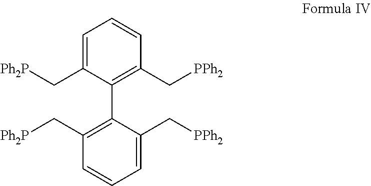

(c) monitoring the levels of total polyphosphorous compounds and polyphosphine ligand and
(d) adjusting the feed rate of the polyphosphine ligand to maintain total polyphosphorous compounds to a set value relative to the moles of transition metal.

In some embodiments, the transition metal is rhodium and the monophosphine is triphenylphosphine. These and other embodiments are discussed in more detail in the Detailed Description below.

### DETAILED DESCRIPTION

All references to the Periodic Table of the Elements and the various groups therein are to the version published in the CRC Handbook of Chemistry and Physics, 72nd Ed. (1991-1992) CRC Press, on page I-11. Unless stated to the contrary, or implicit from the context, all parts and percentages are based on weight and all test methods are current as of the filing date of this application. For purposes of United States patent practice, the contents of any referenced patent, patent application or publication are incorporated by reference in their entirety (or its equivalent US version is so incorporated by reference) especially with respect to the disclosure of definitions (to the extent not inconsistent with any definitions specifically provided in this disclosure) and general knowledge in the art.

As used herein, “a,” “an,” “the,” “at least one,” and “one or more” are used interchangeably. The terms “comprises,” “includes,” and variations thereof do not have a limiting meaning where these terms appear in the description and claims. Also herein, the recitations of numerical ranges by endpoints include all numbers subsumed in that range (e.g., 1 to 5 includes 1, 1.5, 2, 2.75, 3, 3.80, 4, 5, etc.). For the purposes of the invention, it is to be understood, consistent with what one of ordinary skill in the art would understand, that a numerical range is intended to include and support all possible sub-ranges that are included in that range. For example, the range from 1 to 100 is intended to convey from 1.01 to 100, from 1 to 99.99, from 1.01 to 99.99, from 40 to 60, from 1 to 55, etc. Also herein, the recitations of numerical ranges and/or numerical values, including such recitations in the claims, can be read to include the term “about.” In such instances the term “about” refers to numerical ranges and/or numerical values that are substantially the same as those recited herein.

As used herein, the term “ppmw” means parts per million by weight.

For purposes of this invention, the term “hydrocarbon” is contemplated to include all permissible compounds having at least one hydrogen and one carbon atom. Such permissible compounds may also have one or more heteroatoms. In a broad aspect, the permissible hydrocarbons include acyclic (with or without heteroatoms) and cyclic, branched and unbranched, carbocyclic and heterocyclic, aromatic and nonaromatic organic compounds that can be substituted or unsubstituted.

As used herein, the term “substituted” is contemplated to include all permissible substituents of organic compounds unless otherwise indicated. In a broad aspect, the permissible substituents include acyclic and cyclic, branched and unbranched, carbocyclic and heterocyclic, aromatic and nonaromatic substituents of organic compounds. Illustrative substituents include, for example, alkyl, alkyloxy, aryl, aryloxy, hydroxyalkyl, aminoalkyl, in which the number of carbons can range from 1 to 20 or more, preferably from 1 to 12, as well as hydroxy, halo, and amino. The permissible substituents can be one or more and the same or different for appropriate organic compounds. This invention is not intended to be limited in any manner by the permissible substituents of organic compounds.

As used herein, the term “hydroformylation” is contemplated to include, but is not limited to, all hydroformylation processes that involve converting one or more substituted or unsubstituted olefinic compounds or a reaction mixture comprising one or more substituted or unsubstituted olefinic compounds to one or more substituted or unsubstituted aldehydes or a reaction mixture comprising one or more substituted or unsubstituted aldehydes. The aldehydes may be asymmetric or non-asymmetric.

The terms “reaction fluid,” “reaction medium” and “catalyst solution” are used interchangeably herein, and may include, but are not limited to, a mixture comprising: (a) a transition metal-monophosphine complex catalyst (e.g., a rhodium-triphenylphosphine complex catalyst), (b) a transition metal-polyphosphine ligand complex catalyst (e.g., a rhodium-polyphosphine ligand complex catalyst), (c) free monophosphine (e.g., triphenylphosphine), (d) free polyphosphine, (e) aldehyde products formed in the reaction including condensation products derived therefrom (“heavies”), (f) unreacted reactants, (g) a solvent for said transition metal complex catalysts and said free phosphine ligands, and optionally (h) monophosphine ligand and polyphosphine ligand degradation products. The reaction fluid can encompass, but is not limited to, (a) a fluid in a reaction zone, (b) a fluid stream on its way to a separation zone, (c) a fluid in a separation zone, (d) a recycle stream, (e) a fluid withdrawn from a reaction zone or separation zone, (f) a fluid in an external cooler, and (g) ligand degradation products.

As used herein the term “polyphosphine ligand” is contemplated to comprise compounds as described in Formula I with three or more P(III) phosphine moieties capable of binding to the catalytic metal. In one embodiment, the polyphosphine ligand is a tetraphosphine as described in Formula III. The polyphosphine ligand is preferably a custom-manufactured compound deliberately added to the process.

As used herein the terms “ligand degradation” and “ligand decomposition” are used interchangeably and are contemplated to comprise chemical transformations of polyphosphine ligand or polydentate ligand degradation products within the reaction fluid. In some embodiments ligand degradation includes chemical transformation of triarylphosphines which have been added to the process. Such chemical transformations include but are not limited to oxidation of a phosphine moiety and rhodium-promoted side reactions (e.g., aryl-alkyl group exchange, aryl group cleavage), and the like.

As used herein the term “entrainment” is contemplated to comprise the physical egress of catalyst solution from the system. Such egress may include but is not limited to mists and droplets carried into the product stream in a product-catalyst separation zone.

As used herein the terms “ligand consumption” and “ligand consumed” are used interchangeably and are contemplated to comprise a decline in ligand concentration within the catalyst solution. Ligand concentrations may decline due to a plurality of causes, including but not limited to ligand degradation and entrainment. Examples of polyphosphine degradation include oxidation and P-C cleavage processes such as described for triarylphosphines in Abatjoglou A G, Billig E, Bryant D R Mechanism of rhodium-promoted triphenylphosphine reaction in, hydroformylation processes, Organometallics (1984) 3:923-926. Without being bound by theory, and not intending to present an exhaustive list, potential degradation products of a typical tetraphosphine ligand (Formula IV described below) are shown below:

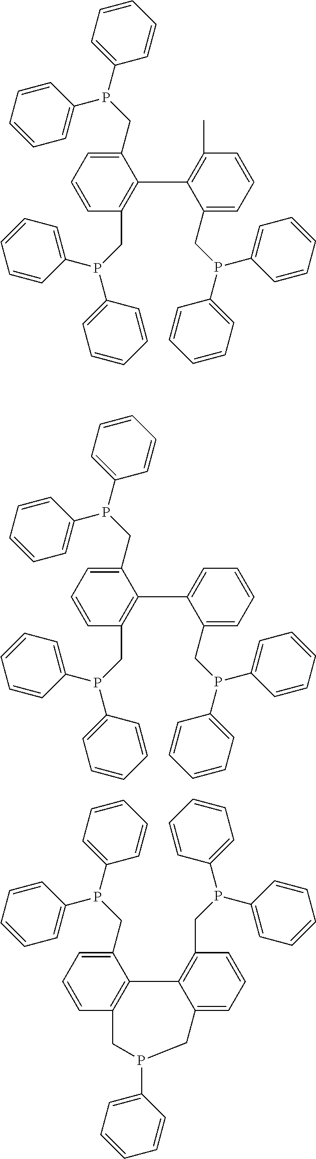

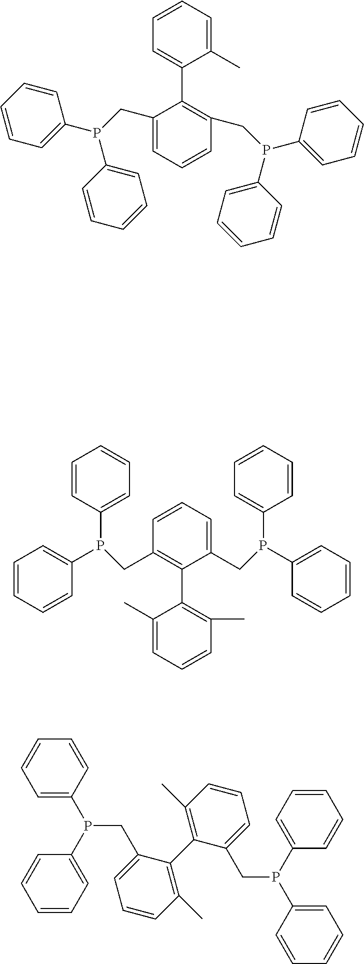 [SMILES: Cc1ccccc1-c1c(CP(c2ccccc2)c2ccccc2)cccc1CP(c1ccccc1)c1ccccc1 ; Cc1cccc(C)c1-c1c(CP(c2ccccc2)c2ccccc2)cccc1CP(c1ccccc1)c1ccccc1 ; Cc1cccc(CP(c2ccccc2)c2ccccc2)c1-c1c(C)cccc1CP(c1ccccc1)c1ccccc1]

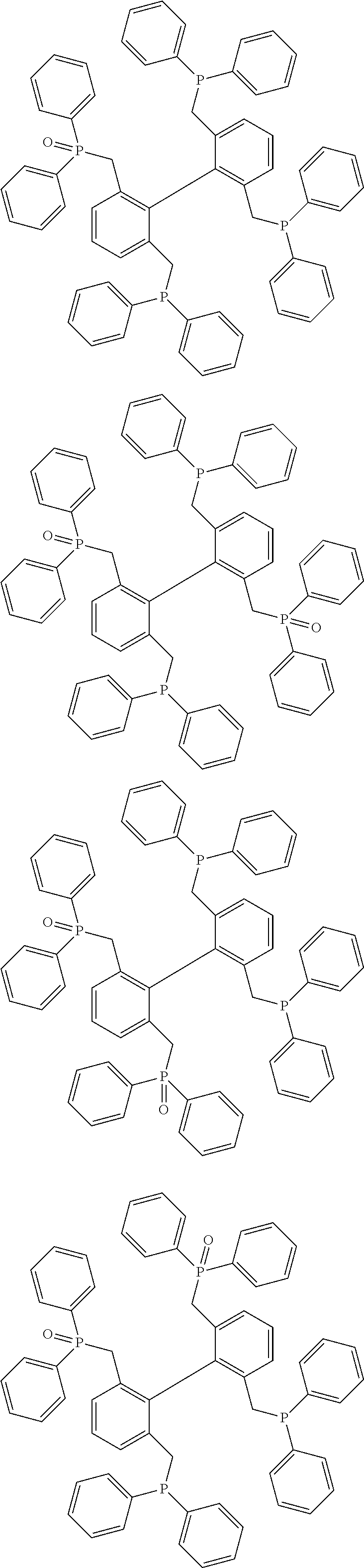

As used herein the term “polydentate ligand degradation products” is contemplated to include all compounds comprising at least two P(III) phosphine moieties which result from ligand degradation of the polyphosphine ligand as well as subsequent further degradation of the initial polydentate ligand degradation products.

As used herein the term “polydentate ligand degradation product consumption rate” is contemplated to include the rate at which the concentration of polydentate ligand degradation products declines in the system. The concentration of polydentate ligand degradation products may decline due to a plurality of causes, including but not limited to ligand degradation and entrainment.

As used herein the term “monodentate ligand degradation product” is contemplated to include all compounds comprising one phosphorous moiety which result from ligand degradation of the polyphosphine ligand. In some embodiments monodentate ligand degradation products may be derived from chemical transformation of triarylphosphine which has been added to the catalyst solution.

The term “triarylphosphine” is contemplated to include compounds as described in Formula II which are optionally added to the process in some embodiments.

As used herein, the term “total polyphosphorous compounds” is contemplated to comprise both polyphosphine ligand (Formula I) and polydentate ligand degradation products (Formula I degradation products).

As used herein, the term “polyphosphine ligand feed rate” is contemplated to comprise the rate at which polyphosphine ligand must be added to offset the polyphosphine ligand consumption and thereby maintain the target concentration in the process.

The term “polyphosphine ligand usage rate” is contemplated to comprise a material balance of polyphosphine ligand within the process during a given period. For example, the polyphosphine ligand concentration at the start of the period in question is determined via analysis, and the polyphosphine ligand feed rate during the period is measured; analysis of the process fluid at the end of the period to determine the concentration of polyphosphine ligand still present in the unit will allow the amount that has been consumed to be calculated. In one embodiment, polyphosphine ligand usage is expressed as the mass of polyphosphine ligand consumed per liter of process volume per unit time (e.g., grams of ligand per liter of process fluid per day; g/L-day).

As used herein the terms “rhodium complex”, “rhodium complex catalyst”, and “catalyst complex” are used interchangeably and are contemplated to comprise at least one rhodium atom with ligands bound or coordinated via electron interaction. Examples of such ligands include but are not limited to monophosphine ligand (if present), polyphosphine ligand, polydentate ligand degradation products, carbon monoxide, olefin (e.g., propylene) and hydrogen. In some embodiments triarylphosphines are added to the process as a ligand.

As used herein, the term “free ligand” is contemplated to include but not limited to monophosphine ligand degradation products, polyphosphine ligand, polydentate ligand degradation products that are not bound or coordinated to rhodium. In some embodiments free ligands will comprise triarylphosphines not bound or coordinated to rhodium.

As used herein the terms “deactivation” and “catalyst deactivation” are used interchangeably and are contemplated to comprise a decline in the hydroformylation rate over time. For example, the rate of aldehyde production (e.g., pounds of product per hour measured by determining the amount of aldehyde in a stream leaving the reactor at a constant flow using gas chromatography) at comparable process conditions is measured regularly over a period of weeks. A decline in the amount of aldehyde being produced would indicate catalyst deactivation.

The concentration of polyphosphine ligand and polydentate ligand degradation products in the reaction fluid should be measured periodically (e.g., daily, several times a week, etc.). High Performance Liquid Chromatography (HPLC) is a preferred method of measurement.

It is important that the analytical method for determining ligand concentration be capable of detecting and quantifying both the polyphosphine ligand and polydentate ligand degradation products such that the sum is readily calculated. The method need not be able to differentiate the exact structure of the degradation product (e.g., which of the phosphines has been oxidized, just that one was oxidized is sufficient). Calibration of HPLC analysis can be done using HPLC-MS techniques wherein the mass spectrum of the peaks can be used to elucidate the degradation product or preparation-scale HPLC can be used to separate the degradation products and mass spectroscopy and ³¹P NMR techniques can further confirm degradation product structures. Generating degraded ligand off-line for use in calibration of the HPLC is a preferred method. Phosphorous NMR techniques may also be used.

The catalytic metal concentration can be measured by common methods such as atomic absorption (AA), inductively coupled plasma (ICP), or X-ray fluorescence (XRF).

The N/I ratio is normally determined by performing gas chromatography (GC) analyses of the product aldehyde; such techniques are well known in the art.

The polyphosphine ligand addition may be continuous (i.e., a slow, roughly continuous feed of ligand) or batchwise. Preferably the polyphosphine ligand is dissolved in a suitable solvent such as described herein but most preferably dissolved in the product aldehyde.

The polyphosphine ligand feed rate is adjusted to achieve the target polyphosphine ligand to catalytic metal ratio. An appropriate feed rate may be calculated based on the current concentration of polyphosphine ligand in the system along with the polyphosphine ligand usage rate. For example, if the target concentration of polyphosphine ligand is higher than the current concentration, the polyphosphine ligand feed rate should exceed the polyphosphine ligand usage rate. Conversely if the target polydentate ligand concentration is lower than the current concentration, the polydentate ligand feed rate should be less than the usage rate.

Likewise, the total polyphosphorous compound:catalytic metal ratio is maintained at the target level by adjusting the feed rate of the polyphosphine ligand.

The method of ligand addition is not narrowly critical for this invention if the concentration of polyphosphine ligand is kept within the target range. In practice, there will be some unavoidable fluctuation in the addition rate due to a plurality of causes, including analytical or equipment variation, and the like. Likewise, batch-mode additions may generate a “saw-tooth” pattern, but in general it is preferred that the concentration of polyphosphine ligand be kept within a relatively narrow range. Polyphosphine ligand usage will also vary somewhat as a function of the actual plant equipment, operating parameters (and changes thereto such as reactor temperature and reagent partial pressures) but a skilled person in the art will be able to adjust the polyphosphine ligand addition rate to maintain their target polyphosphine ligand to catalytic metal ratio.

It has surprisingly been discovered that maintaining an advantageous balance of total polyphosphorous compounds comprised of polyphosphine ligand and polydentate ligand degradation products in the catalyst solution will allow the desired hydroformylation performance to be achieved, while at the same time reducing the usage rate of polyphosphine ligand.

Without wishing to be bound by theory, it is proposed that the polydentate ligand degradation products promote hydroformylation sufficiently to allow the polyphosphine ligand target concentration to be reduced beyond what is normally maintained in hydroformylation processes comprising rhodium and chelating ligands (e.g., less than one equivalent relative to rhodium). The degradation rate of the polyphosphine ligand is concentration dependent; therefore, lowering its concentration will slow the polyphosphine ligand usage. Because the polydentate ligand degradation products are derived from polyphosphine degradation, the formation rate of these compounds will slow as polyphosphine ligand degradation declines. Polydentate ligand degradation products will also continue to be consumed however, primarily due to rhodium-promoted side reactions. If the addition of polyphosphine ligand is halted or reduced too much, the concentration of total polyphosphorous compounds in the catalyst solution will eventually be insufficient to provide the desired catalysis. Therefore, action must be taken to achieve the result of the current invention, which is advantaged hydroformylation performance and lower polyphosphine ligand usage.

To avoid loss of the transition metal to inactive forms such as clusters or plating out on reactor surfaces, the total moles of chelating ligands (e.g., total polyphosphorous compounds) and optional triarylphosphine must at least equal and preferably exceed the moles of transition metal.

In one embodiment, the polyphosphine addition rate is adjusted to maintain the total polyphosphorous compounds:rhodium ratio in the catalyst solution at about 1.5:1. In another embodiment the target for the total polyphosphorous compound:rhodium ratio is about 2:1. In another embodiment, the target for the total polyphosphorous compound:rhodium ratio is about 3:1.

In one embodiment the total polyphosphorous compounds comprise at least one polyphosphine and at least one polyphosphine degradation product wherein there is less than 1 equivalent of polyphosphine ligand relative to rhodium. In another embodiment the polyphosphine ligand:rhodium ratio is ≥0.25:1.

In one embodiment, the N/I of the product resulting from the process is greater than 12, or even 15.

Catalyst performance will provide an indirect measurement of the relative concentrations of polyphosphine ligand and polydentate ligand degradation compounds which comprise the total polyphosphorous compounds. A decline in the N/I could indicate that the polyphosphine ligand:rhodium ratio and/or the total polyphosphorous compound concentration has fallen below target; this can be remedied by increasing the polyphosphine ligand feed rate.

In one aspect, the present invention relates to a hydroformylation catalyst composition comprising:
(a) a polyphosphine having the following structure:

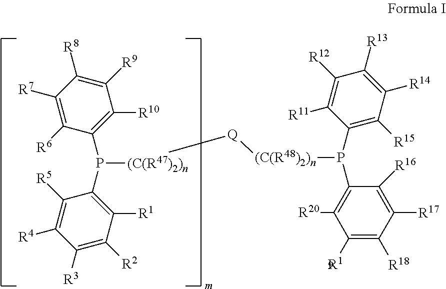

wherein each P is a phosphorous atom, and each of R¹-R²⁰ are independently hydrogen, a C1 to C8 alkyl group, a C1 to C8 alkoxyl group, an aryl group, an alkaryl group, or a halogen, R⁴⁷ and R⁴⁸ are independently hydrogen, a C1 to C8 alkyl group, or a C1 to C8 substituted alkyl group, n is 0 or 1, in is 2 or higher, and Q is a m+1 valent organic radical, wherein R⁵ and R⁶ and/or R¹⁵ and R¹⁶ may be links to form bridged structures, and
(b) polyphosphine degradation products derived from the polyphosphine of Formula I wherein the molar ratio of the polyphosphine degradation products to Formula I is at least 0.25:1 and
(c) a Group 8, 9 and 10 transition metal
(d) a solvent comprising the product aldehyde and aldehyde condensation products
(e), optionally; a monophosphine having the following structure:

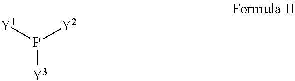

wherein P is a phosphorous atom, and each of Y¹-Y³ are independently an aryl group or a substituted aryl group,
wherein that the sum of the Formula I and Formula I degradation products relative to the moles of transition metal is greater than 1.1:1 and the Formula I to transition metal mole ratio is less than 1:1.

Another aspect of the present invention are general methods for slowing polyphosphine ligand usage in a hydroformylation process. The catalyst includes a catalyst composition derived from a transition metal (e.g., rhodium), a polyphosphine ligand, and the polyphosphine ligand degradation products such that the polyphosphine degradation products are present in sufficient amount to enable a reduction of the polyphosphine ligand feed rate.

In one embodiment, the present invention is directed to a method to reduce polyphosphine ligand usage in a hydroformylation process comprising:
(a) contacting an olefin with carbon monoxide, hydrogen and a catalyst, the catalyst comprising (A) a transition metal; (B) a tetraphosphine having the following structure:

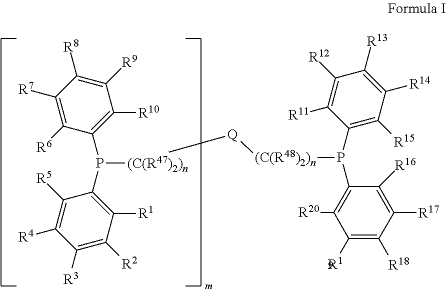

wherein each P is a phosphorous atom, and each of R¹-R²⁰ are independently hydrogen, a C1 to C8 alkyl group, an aryl group, an alkaryl group, or a halogen, R⁴⁷ and R⁴⁸ are independently hydrogen, a C1 to C8 alkyl group, or a C1 to C8 substituted alkyl group, wherein R⁵ and R⁶ and R¹⁵ and R¹⁶ may be links to form bridged structures, each n is zero or one, and m is 2 or higher and,
(b) optionally, a triarylphosphine with the following structure:

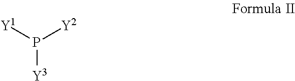

wherein P is a phosphorous atom, and each of Y¹-Y³ are independently an aryl group or a substituted aryl group, the contacting conducted in one or more reaction zones and at hydroformylation conditions; and
(c) monitoring the levels of total polyphosphorous compounds and adjusting the feed rate of the polyphosphine ligand to maintain the concentration of total polyphosphorous compounds to a target value relative to the moles of transition metal of greater than 1.25:1 and wherein the polyphosphine ligand to catalyst metal molar ratio is less than 1:1.

It may be advantageous in some embodiments to ensure that the polyphosphine ligand to transition metal ratio is greater than 0.25:1.

In one embodiment, the present invention is directed to a method to reduce polyphosphine ligand usage in a hydroformylation process comprising:
(a) contacting an olefin with carbon monoxide, hydrogen and a catalyst, the catalyst comprising (A) a transition metal; (B) a tetraphosphine having the following structure:

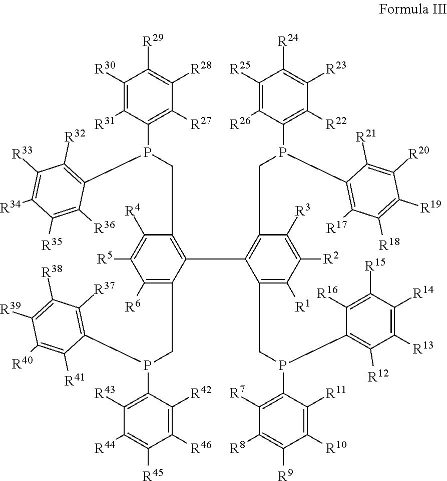

wherein each P is a phosphorous atom, and each of R¹-R⁴⁶ are independently hydrogen, a C1 to C8 alkyl group, an aryl group, an alkaryl group, or a halogen, and,
(b) optionally, a triarylphosphine having the following structure:

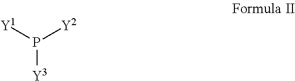

wherein P is a phosphorous atom, and each of Y¹-Y³ are independently an aryl group or a substituted aryl group, the contacting conducted in one or more reaction zones and at hydroformylation conditions; and
(b) monitoring the levels of total polyphosphorous compounds and adjusting the feed rate of the polyphosphine ligand to maintain the concentration of total polyphosphorous compounds to a target value relative to the moles of transition metal of greater than 1.25:1 and wherein the polyphosphine ligand to catalyst metal molar ratio is less than 1:1.

In one embodiment, the present invention is directed to a method to reduce polyphosphine ligand usage in a hydroformylation process comprising:
(a) contacting an olefin with carbon monoxide, hydrogen and a catalyst, the catalyst comprising (a) a transition metal; (b) a tetraphosphine having the following structure:

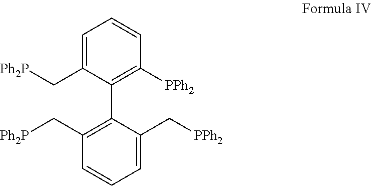

And,
(c) a triarylphosphine having the following structure:

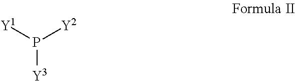

wherein P is a phosphorous atom, and each of Y¹-Y³ are independently an aryl group or a substituted aryl group, the contacting conducted in one or more reaction zones and at hydroformylation conditions; and
(b) monitoring the levels of total polyphosphorous compounds and adjusting the feed rate of the polyphosphine ligand to maintain the concentration of total polyphosphorous compounds to a target value relative to the moles of transition metal of greater than 1.25:1 and wherein the polyphosphine ligand to catalyst metal molar ratio is less than 1:1.

The transition metal, in some embodiments, comprises rhodium. In some embodiments, the olefin is propylene. In some embodiments, triarylphosphine is 3, 10, or even 20 moles of triarylphosphine per mole of transition metal up to 350, 375, or even 400 moles triarylphosphine per mole of transition metal.

The triarylphosphine, in some embodiments, is one or more of the following: triphenylphosphine, tris(o-tolyl)phosphine, trinaphthylphosphine, tri(p-methoxyphenyl) phosphine, and tri(m-chlorophenyl)-phosphine. The triarylphosphine is triphenylphosphine in some embodiments. In some embodiments, the catalyst comprises a mixture of different species of triarylphosphines.

In some embodiments, each of R¹-R⁴⁶ in Formula III for the polyphosphine are hydrogen. In some embodiments the catalyst comprises one or more of the following tetraphosphines:

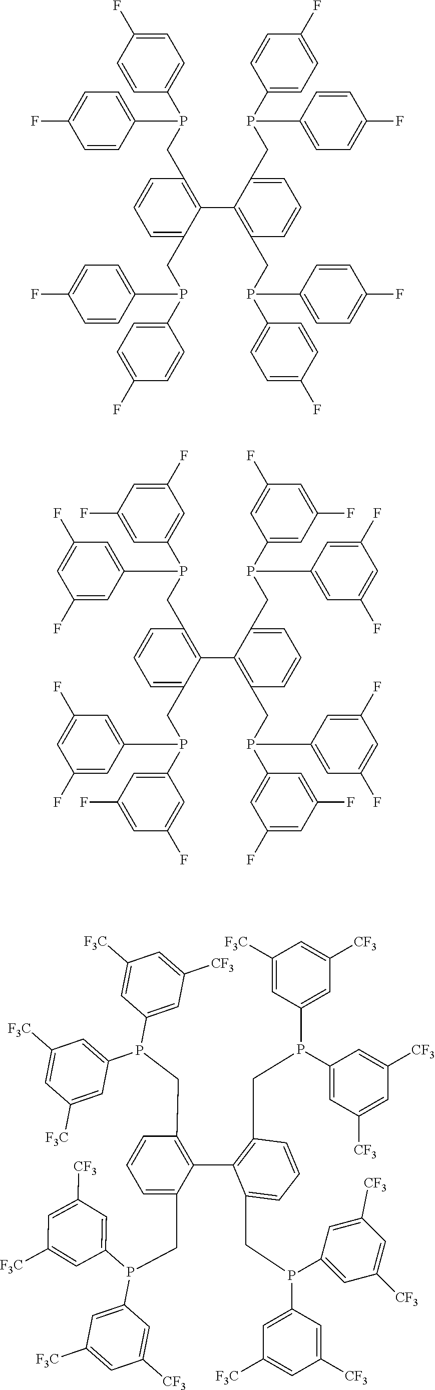

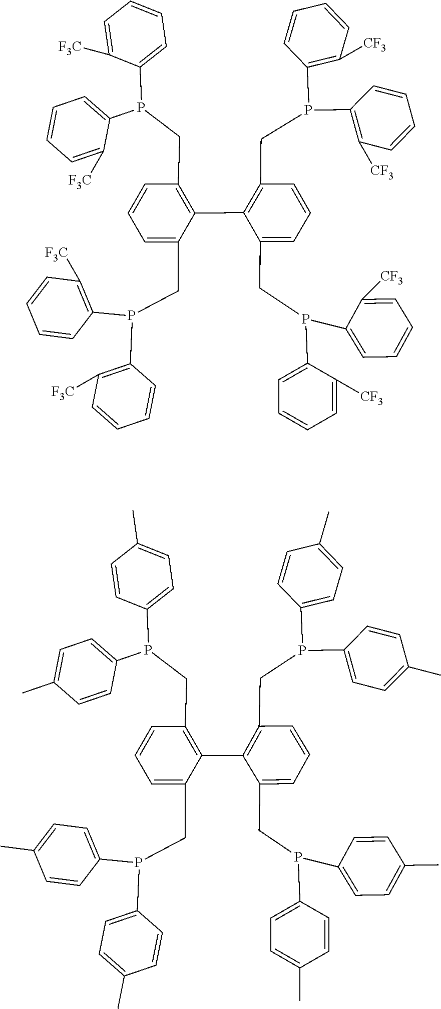

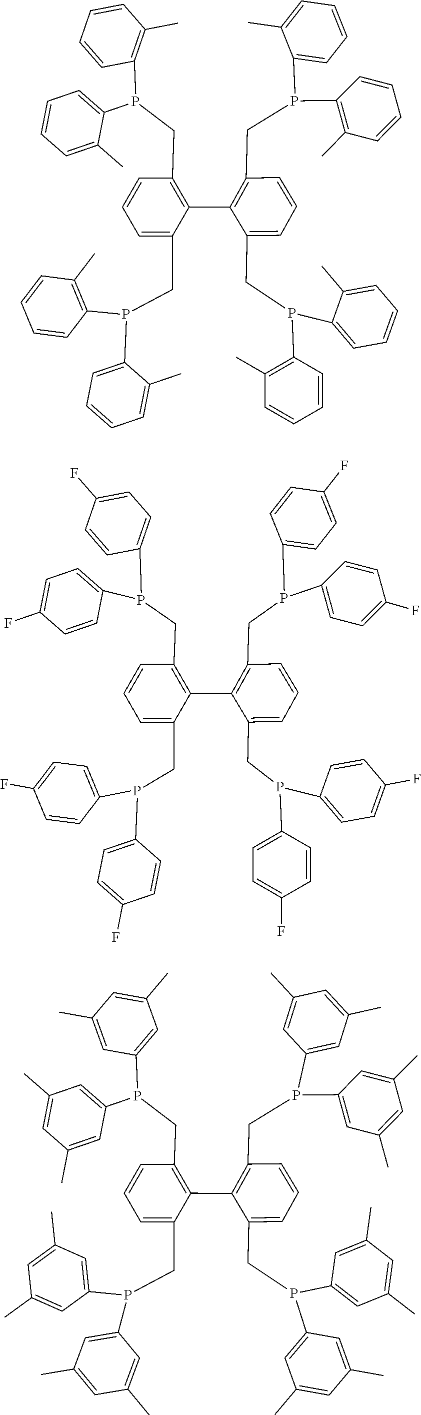

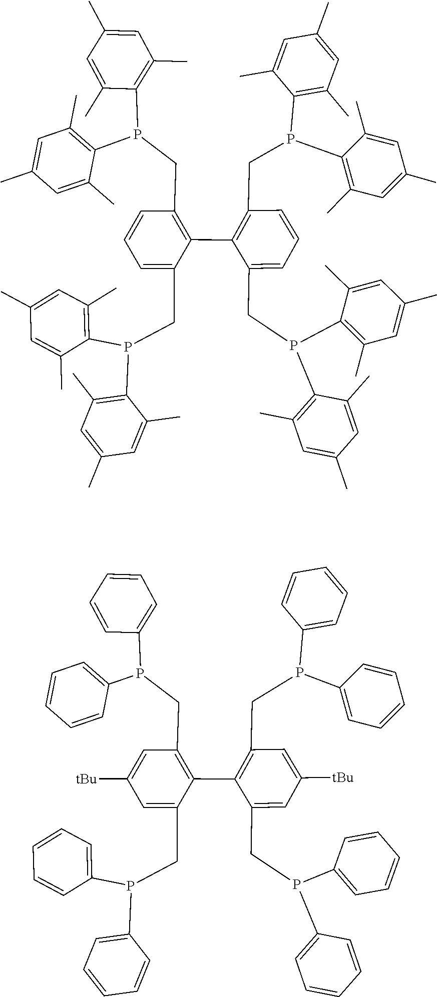

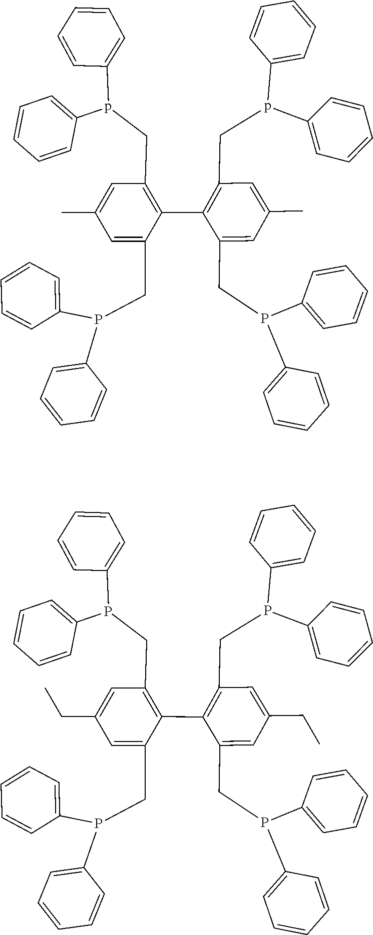

In some embodiments, the transition metal comprises rhodium, the triaryl phosphine of Formula II is triphenylphosphine, each of R¹-R⁴⁶ in Formula III are hydrogen, and the olefin comprises propylene.

Hydrogen and carbon monoxide may be obtained from any suitable source, including petroleum cracking and refinery operations. Syngas (from synthesis gas) is the name given to a gas mixture that contains varying amounts of CO and H₂. Production methods are well known. Hydrogen and CO typically are the main components of syngas, but syngas may contain CO₂ and inert gases such as N₂ and Ar. The molar ratio of H₂ to CO varies greatly but generally ranges from 1:100 to 100:1 and preferably between 1:10 and 10:1. Syngas is commercially available and is often used as a fuel source or as an intermediate to produce other chemicals. The most preferred H₂:CO molar ratio for chemical production is between 3:1 and 1:3 and usually is targeted to be between about 1:2 and 2:1 for most hydroformylation applications. Syngas mixtures are a preferred source of hydrogen and CO.

The olefinic starting material reactants that may be employed in the hydroformylation reactions encompassed by of this invention can be terminally or internally unsaturated and be of straight-chain, branched-chain or cyclic structure. Such olefins can contain from 2 to 20 carbon atoms (C2-C20) and may contain one or more ethylenic unsaturated groups. Moreover, such olefins may contain groups or substituents which do not essentially adversely interfere with the hydroformylation process such as carbonyl, carbonyloxy, oxy, hydroxy, oxycarbonyl, halogen, alkoxy, aryl, alkyl, haloalkyl, and the like. Illustrative olefinic unsaturated compounds include alpha olefins, internal olefins, alkyl alkenoates, alkenyl alkanoates, alkenyl alkyl ethers, alkenols, and the like, e.g. ethylene, propylene, 1-butene, 1-pentene, 1-hexene, 1-octene, 1-decene, 1,-dodecene, 1-octadecene, 2-butene, isobutylene, 2-methylbutene, 2-hexene, 3-hexene, 2-heptene, cyclohexene, propylene dimers, propylene trimers, propylene tetramers, butene dimers, butene trimers, 2-ethyl-1-hexene, styrene, 3-phenyl-1-propene, 1,4-hexadiene, 1,7-octadiene, 3-cyclohexyl-1-butene, and the like. Of course, it is understood that mixtures of different olefinic starting materials can be employed, if desired. Embodiments of the present invention can be particularly useful in the hydroformylation of C3 and higher olefins. Thus, in some embodiments, the olefinic unsaturated starting materials are alpha olefins containing from 3 to 20 carbon atoms, and internal olefins containing from 3 to 20 carbon atoms as well as starting material mixtures of such alpha olefins and internal olefins. In some embodiments the olefin comprises vinyl silanes and vinyl siloxanes.

A solvent advantageously is employed in the hydroformylation process. Any suitable solvent that does not unduly interfere with the hydroformylation process can be used. By way of illustration, suitable solvents for rhodium catalyzed hydroformylation processes include those disclosed, for example, in U.S. Pat. Nos. 3,527,809; 4,148,830; 5,312,996; and 5,929,289. Non-limiting examples of suitable solvents include saturated hydrocarbons (alkanes), aromatic hydrocarbons, ethers, aldehydes, ketones, nitriles, alcohols, esters, and aldehyde condensation products. Specific examples of solvents include: tetraglyme, pentanes, cyclohexane, heptanes, benzene, xylene, toluene, diethyl ether, tetrahydrofuran, butyraldehyde, and benzonitrile. The organic solvent may also contain dissolved water up to the saturation limit. Illustrative preferred solvents include ketones (e.g. acetone and methylethyl ketone), esters (e.g. ethyl acetate, di-2-ethylhexyl phthalate, 2,2,4-trimethyl-1,3-pentanediol monoisobutyrate), hydrocarbons (e.g. toluene), nitrohydrocarbons (e.g., nitrobenzene), ethers (e.g., tetrahydrofuran (THF)) and sulfolane. In rhodium catalyzed hydroformylation processes, it may be preferred to employ, as a primary solvent, aldehyde compounds corresponding to the aldehyde products desired to be produced and/or higher boiling aldehyde liquid condensation by-products, for example, as might be produced in situ during the hydroformylation process, as described for example in U.S. Pat. Nos. 4,148,830 and 4,247,486. The primary solvent will normally eventually comprise both aldehyde products and higher boiling aldehyde liquid condensation by-products (“heavies”), due to the nature of the continuous process. The amount of solvent is not especially critical and need only be sufficient to provide the reaction medium with the desired amount of transition metal concentration. Typically, the amount of solvent ranges from about 5 percent to about 95 percent by weight, based on the total weight of the reaction fluid. Mixtures of solvents may be employed.

The catalyst used in methods of the present invention comprises a transition metal and a polyphosphine ligand. In certain particularly useful embodiments, the catalyst comprises rhodium and a polyphosphine ligand. The most desirable catalyst is free of metal-bound halogens such as chlorine, and contains hydrogen, carbon monoxide and polyphosphine ligand complexed with the transition metal, preferably rhodium, to produce a catalyst soluble in the liquid phase and stable under the conditions of the reaction. The transition metal can include Group 8, 9 and 10 metals selected from rhodium (Rh), cobalt (Co), iridium (Ir), ruthenium (Ru), iron (Fe), nickel (Ni), palladium (Pd), platinum (Pt), osmium (Os) and mixtures thereof, with preferred metals being rhodium, cobalt, iridium and ruthenium, more preferably rhodium, cobalt and ruthenium, especially rhodium. The number of available coordination sites on such metals is well known in the art but the exact coordination mode for the polyphosphines and optional monophosphine is not known. Thus, the catalytic species, which may comprise a complex catalyst mixture, may comprise monomeric, dimeric or higher nuclearity forms, which are preferably characterized by at least one organophosphine-containing molecule complexed per one molecule of metal, e.g., rhodium. For instance, it is considered that the catalytic species of the preferred catalyst employed in a hydroformylation reaction may be complexed with carbon monoxide and hydrogen in addition to the organophosphine ligands in view of the carbon monoxide and hydrogen gas employed by the hydroformylation reaction. In certain preferred embodiments, the transition metal is rhodium. Rhodium can be introduced into the liquid phase as a preformed catalyst, e.g., a stable crystalline solid, rhodium hydridocarbonyl-tris(triphenyl phosphine), RhH(CO)(PPh₃)₃ which conveniently introduces one equivalent of a triarylphosphine. The rhodium can also be introduced to the liquid body as a precursor form which is converted in situ into the catalyst. Examples of such precursor form are rhodium carbonyl triphenylphosphine acetylacetonate, Rh₂O₃, Rh₄(CO)₁₂, Rh₆(CO)₁₆, and rhodium dicarbonyl acetylacetonate. Both the catalyst compounds which will provide active species in the reaction medium, and their preparation are known by art, see Brown et al., Journal of the Chemical Society, 1970, pp. 2753-2764.

In general, the optimum catalyst concentration depends on the concentration of the alpha-olefin, such as propylene. For example, the higher the propylene concentration the lower usually will be the catalyst concentration needed to achieve a given conversion rate to aldehyde products in a given size reactor. Recognizing that partial pressures and concentration are related, the use of higher propylene partial pressure leads to an increased proportion of propylene in the “off gas” from the liquid body. Since it may be necessary to purge part of the gas stream from the product recovery zone before recycling to the liquid body in order to remove a portion of the propane or other inert gases which may be present, the higher the propylene content of the “off gas”, the more propylene that will be lost in the propane purge stream. Thus, it is necessary to balance the economic value of the propylene lost in the propane purge stream against the capital savings associated with lower catalyst concentration.

The rhodium complex catalysts are preferably in a homogeneous form. For instance, pre-formed rhodium hydrido-carbonyl-phosphine ligand catalysts may be prepared and introduced into a hydroformylation reaction mixture. Preferably, the rhodium-phosphine ligand complex catalysts can be derived from a rhodium catalyst precursor that may be introduced into the reaction medium for in situ formation of the active catalyst. For example, rhodium catalyst precursors such as rhodium dicarbonyl acetylacetonate, Rh₂O₃, Rh₄(CO)₁₂, Rh₆(CO)₁₆, Rh(NO₃)₃ and the like may be introduced into the reaction mixture along with the polyphosphine for the in-situ formation of the active catalyst. In a preferred embodiment, rhodium dicarbonyl acetylacetonate is employed as a rhodium precursor and combined with polyphosphine in a solvent and introduced into the reactor along with syn gas for the in-situ formation of the active catalyst. Additional polyphosphine ligand and/or optional triarylphosphine may be added as necessary to achieve and maintain the desired concentrations. In any event, it is sufficient that carbon monoxide, hydrogen, and polyphosphine are all ligands that are capable of being complexed with the metal and that an active metal-ligand catalyst is present in the reaction mixture under the conditions used in the hydroformylation reaction.

The amount of rhodium complex catalyst present in the reaction fluid is the minimum amount necessary to produce the desired production rate. In general, rhodium concentrations in the range of from 50 ppmw to 1200 ppmw, calculated as free metal in the reaction fluid, should be sufficient for most processes, while it is generally preferred to employ from 100 to 800 ppmw of metal, and more preferably from 150 to 500 ppmw of rhodium.

Any manner may be used to start-up and establish a consistent concentration profile of tetraphosphine ligand and total polyphosphorous compounds.

Accordingly, the rhodium-ligand complex catalysts in the reaction fluid advantageously comprise rhodium complexed with carbon monoxide and polyphosphine ligand. In one embodiment, mixtures of rhodium-ligand complexes are employed. For example, the catalyst additionally comprises rhodium complexed with carbon monoxide and polyphosphine ligand in a chelated and/or non-chelated fashion. In one preferred embodiment, the rhodium precursor is Rh(triphenylphosphine)(acetylacetonate)(CO) which introduces at least a portion of triphenylphosphine to the system.

As noted herein, the polyphosphine ligand and the polydentate ligand degradation products derived therefrom, (and optionally the monophosphine) are lost or degraded over time by a variety of mechanisms. For commercial operations, the desired concentrations must be maintained by periodic or continuous additions. To do so, the concentrations of organophosphorous ligands in the reaction fluid are routinely measured by one or more analytical techniques. Unless otherwise indicated herein, when referring to the amount of ligand in a reaction, the ligand concentration is determined by HPLC such as described in the Examples. Ligand concentrations in such analyses are often reported as weight percent; thus, it is often convenient to use these units for continuous operation.

In addition to the rhodium complex catalyst, free triarylphosphine may also be present in the reaction fluid and may also be present in a catalyst composition prior to being provided to a reactor. The significance of free ligand is taught in U.S. Pat. No. 3,527,809, GB 1,338,225, and Brown et al., supra., pages 2759 and 2761. In some embodiments, the hydroformylation process of this invention may involve from 30 moles of free (uncomplexed) triarylphosphine per mole of rhodium in the reaction medium, preferably above 40 moles triarylphosphine per mole rhodium, and most preferably above 50 moles triarylphosphine per mole rhodium up to a maximum of 400 moles free triarylphosphine per mole of rhodium.

The tetraphosphine compounds that may serve as the ligands in embodiments of the present invention are compounds of Formula III:

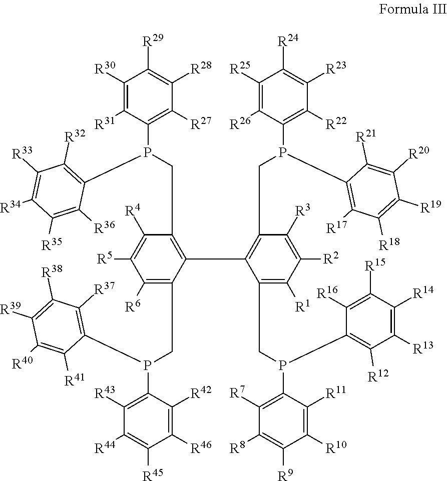

wherein each P is a phosphorous atom, and each of R¹-R⁴⁶ are independently hydrogen, a C1 to C8 alkyl group, an aryl group, an alkaryl group, a haloalkyl group, or a halogen and wherein (R⁵ and R⁶), (R¹⁵ and R¹⁶), (R²⁵ and R²⁶), and (R³⁵ and R³⁶) can be bonds. In a preferred embodiment, each of R¹-R⁴⁶ is hydrogen. Other examples of polyphosphines that can be used in some embodiments are described elsewhere in the present specification.

Mixtures of polyphosphines can be used in some embodiments.

Hydroformylation processes, and conditions for their operation, are well known. In a typical embodiment, an olefin (e.g., propylene) is hydroformylated in a continuous or semi-continuous fashion, with the product being separated in a separation zone, and the concentrated catalyst solution being recycled back into one or more reactors. The recycle procedure generally involves withdrawing a portion of the liquid reaction medium containing the catalyst and aldehyde product from the hydroformylation reactor, i.e., reaction zone, either continuously or intermittently, and recovering the aldehyde product therefrom by use of a composite membrane, such as disclosed in U.S. Pat. Nos. 5,430,194 and 5,681,473, or by the more conventional and preferred method of distilling it (i.e. vaporization), in one or more stages under normal, reduced or elevated pressure, as appropriate, in a separate distillation zone, the non-volatilized metal catalyst containing residue being recycled to the reaction zone as disclosed, for example, in U.S. Pat. No. 5,288,918. Condensation of the volatilized materials, and separation and further recovery thereof, e.g., by further distillation, can be carried out in any conventional manner, the crude aldehyde product can be passed on for further purification and isomer separation, if desired, and any recovered reactants, e.g., olefinic starting material and syngas, can be recycled in any desired manner to the hydroformylation zone (reactor). The recovered metal catalyst containing retentate of such membrane separation or recovered non-volatilized metal catalyst containing residue of such vaporization separation can be recycled, to the hydroformylation zone (reactor) in any conventional manner desired.

A typical hydroformylation reaction fluid utilizing a rhodium-polyphosphine ligand complex contains at least some amount of four main ingredients or components, i.e., the aldehyde product, a rhodium-polyphosphine ligand complex catalyst, free polyphosphine ligand, and a solvent for said catalyst and said free polyphosphine ligand. The hydroformylation reaction mixture compositions can and normally will contain additional ingredients such as those that have either been deliberately employed in the hydroformylation process or formed in situ during said process. Examples of such additional ingredients include monotriarylphosphines (Formula II), unreacted olefin starting material, carbon monoxide and hydrogen gases, and in situ formed by-products, ligand degradation compounds, and high boiling liquid aldehyde condensation by-products, as well as other inert co-solvent type materials or hydrocarbon additives, if employed.

The hydroformylation reaction conditions employed may vary. For instance, the total gas pressure of hydrogen, carbon monoxide and olefin starting compound of the hydroformylation process may range from 1 to 69,000 kPa. In general, however, it is preferred that the process be operated at a total gas pressure of hydrogen, carbon monoxide and olefin starting compound of less than 14,000 kPa and more preferably less than 3,400 kPa. The minimum total pressure is limited predominantly by the amount of reactants necessary to obtain a desired rate of reaction. The minimum total pressure is limited predominantly by the amount of reactants necessary to obtain a desired rate of reaction. More specifically, the carbon monoxide partial pressure of the hydroformylation process is preferably from 1 to 6,900 kPa, and more preferably from 21 to 5,500 kPa, while the hydrogen partial pressure is preferably from 34 to 3,400 kPa and more preferably from 69 to 2,100 kPa. In general, the molar ratio of gaseous H₂:CO may range from 1:10 to 100:1 or higher, the more preferred molar ratio being from 1:10 to 10:1.

In general, the hydroformylation process may be conducted at any operable reaction temperature. Advantageously, the hydroformylation process is conducted at a reaction temperature from −25° C. to 200° C., preferably from 50° C. to 120° C.

The hydroformylation process may be carried out using one or more suitable reactors such as, for example, a continuous stirred tank reactor (CSTR), Venturi reactor, or a bubble column reactor. The optimum size and shape of the reactor will depend on the type of reactor used. The reaction zone employed may be a single vessel or may comprise two or more discrete vessels. The separation zone employed may be a single vessel or may comprise two or more discrete vessels. The reaction zone(s) and separation zone(s) employed herein may exist in the same vessel or in different vessels. For example, reactive separation techniques such as reactive distillation, and reactive membrane separation may occur in the reaction zone(s).

The hydroformylation process can be conducted with recycle of unconsumed starting materials if desired. The reaction can be conducted in a single reaction zone or in a plurality of reaction zones and in series or in parallel. The reaction steps may be affected by the incremental addition of one of the starting materials to the other. Also, the reaction steps can be combined by the joint addition of the starting materials. The starting materials may be added to each or all of the reaction zones in series. When complete conversion is not desired or not obtainable, the starting materials can be separated from the product, for example by distillation, and the starting materials then recycled back into the reaction zone.

The hydroformylation process may be conducted in either glass lined, stainless steel or similar type reaction equipment. The reaction zone may be fitted with one or more internal and/or external heat exchanger(s) in order to control undue temperature fluctuations, or to prevent any possible “runaway” reaction temperatures.

The hydroformylation process of this invention may be conducted in one or more steps or stages. The exact number of reaction steps or stages will be governed by the best compromise between capital costs and achieving high catalyst selectivity, activity, lifetime and ease of operability, as well as the intrinsic reactivity of the starting materials in question and the stability of the starting materials and the desired reaction product to the reaction conditions. In one embodiment, the hydroformylation process of the current invention may be carried out in a multistage reactor such as described, for example, in U.S. Pat. No. 5,728,893. Such multistage reactors can be designed with internal, physical barriers that create more than one theoretical reactive stage per vessel.

As discussed herein, in a hydroformylation process over time, ligand loss or degradation will occur. In some cases, catalyst deactivation may also be observed. Catalyst deactivation and/or ligand degradation can advantageously be reduced in hydroformylation processes comprising polyphosphine ligands by adding a triarylphosphine (as described further herein) to the reaction zone. The triarylphosphine compounds that may be added to the reaction zone according to some embodiments of the present invention are compounds of Formula II:

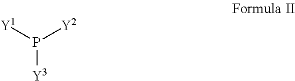

wherein P is a phosphorous atom, and each of Y¹-Y³ are independently an aryl group or a substituted aryl group. Illustrative examples include but are not limited to triphenylphosphine, tris(o-tolyl)phosphine, trinaphthylphosphine, tri(p-methoxyphenyl) phosphine, tri(m-chlorophenyl)-phosphine, and the like. Representative preferred triarylphosphines include those described in U.S. Pat. Nos. 4,283,562 and 5,741,945 (e.g., col. 10, line 57 through col. 13, line 39).

Mixtures of triarylphosphines can be used in some embodiments.

The amount of triarylphosphine that can be added to a reaction zone, in some embodiments, is at least 40 moles of triarylphosphine per mole of transition metal (rhodium). In some embodiments, the amount of triarylphosphine added to a reaction zone is from 30 to 400 moles of triarylphosphine per mole of transition metal (rhodium).

It is generally preferred to carry out the hydroformylation process in a continuous manner. Continuous hydroformylation processes are well known in the art. The continuous process can be carried out in a single pass mode, i.e., wherein a vaporous mixture comprising unreacted olefinic starting material(s) and vaporized aldehyde product is removed from the liquid reaction mixture from whence the aldehyde product is recovered and make-up olefinic starting material(s), carbon monoxide and hydrogen are supplied to the liquid reaction medium for the next single pass through without recycling the unreacted olefinic starting material(s). Such types of recycle procedure are well known in the art and may involve the liquid recycling of the metal-organophosphorous complex catalyst fluid separated from the desired aldehyde reaction product(s), such as disclosed, for example, in U.S. Pat. No. 4,148,830 or a gas recycle procedure such as disclosed, for example, in U.S. Pat. No. 4,247,486, as well as a combination of both a liquid and gas recycle procedure if desired. The most preferred hydroformylation process comprises a continuous liquid catalyst recycle process. Suitable liquid catalyst recycle procedures are disclosed, for example, in U.S. Pat. Nos. 4,668,651; 4,774,361; 5,102,505 and 5,110,990.

In one embodiment, the aldehyde product mixtures may be separated from the other components of the crude reaction mixtures in which the aldehyde mixtures are produced by any suitable method such as, for example, solvent extraction, crystallization, distillation, vaporization, wiped film evaporation, falling film evaporation, phase separation, filtration, or any combination thereof. It may be desired to remove the aldehyde products from the crude reaction mixture as they are formed through the use of trapping agents as described in WO 88/08835. One method for separating the aldehyde mixtures from the other components of the crude reaction mixtures is by membrane separation, which is described, for example in U.S. Pat. Nos. 5,430,194 and 5,681,473.

As indicated above, desired aldehydes may be recovered from the reaction mixtures. For example, the recovery techniques disclosed in U.S. Pat. Nos. 4,148,830 and 4,247,486 can be used. For instance, in a continuous liquid catalyst recycle process the portion of the liquid reaction mixture (containing aldehyde product, catalyst, etc.), i.e., reaction fluid, removed from the reaction zone can be passed to a separation zone, e.g., vaporizer/separator, wherein the desired aldehyde product can be separated via distillation, in one or more stages, under normal, reduced or elevated pressure, from the liquid reaction fluid, condensed and collected in a product receiver, and further purified if desired. The remaining non-volatilized catalyst containing liquid reaction mixture may then be recycled back to the reactor as may, if desired, any other volatile materials, e.g., unreacted olefin, together with any hydrogen and carbon monoxide dissolved in the liquid reaction after separation thereof from the condensed aldehyde product, e.g., by distillation in any conventional manner.

More particularly, distillation and separation of the desired aldehyde product from the metal-organophosphorous complex catalyst containing reaction fluid may take place at any suitable temperature desired. In general, it is preferred that such distillation take place at relatively low temperatures, such as below 150° C., and more preferably at a temperature in the range of from 50° C. to 140° C. In one embodiment, such aldehyde distillation takes place under reduced pressure, e.g., a total gas pressure that is substantially lower than the total gas pressure employed during hydroformylation when low boiling aldehydes (e.g., C₄ to C₆) are involved or under vacuum when high boiling aldehydes (e.g., C₇ or greater) are involved. For instance, a common practice is to subject the liquid reaction product medium removed from the hydroformylation reactor to a pressure reduction so as to volatilize a substantial portion of the unreacted gases dissolved in the liquid medium that now contains a much lower synthesis gas concentration than is present in the reaction medium to the distillation zone, e.g., vaporizer/separator, wherein the desired aldehyde product is distilled. In general, distillation pressures ranging from vacuum pressures on up to total gas pressure of 340 kPa should be sufficient for most purposes.

In one embodiment, flowing gases may be used in the separation zone to facilitate the aldehyde distillation. Such strip gas vaporizers are described for example in U.S. Pat. No. 8,404,903.

The increased concentrations, high temperatures, and low partial pressures that occur in the separation zone may negatively affect the catalyst, both in terms of catalyst deactivation and/or increased ligand usage. As described below in the Examples, an accelerated testing procedure, referred to herein as the block-in procedure, has been devised for demonstrating the impact of the separation zone on the catalyst in order to evaluate various embodiments.

Illustrative non-optically active aldehyde products of hydroformylation processes according to embodiments of the present invention will depend on the olefin used as a reactant and can include e.g., propionaldehyde, n-butyraldehyde, isobutyraldehyde, n-valeraldehyde, 2-methyl 1-butyraldehyde, hexanal, hydroxyhexanal, 2-methyl 1-heptanal, nonanal, 2-methyl-1-octanal, decanal, adipaldehyde, 2-methylglutaraldehyde, 2-methyladipaldehyde, 3-hydroxypropionaldehyde, 6-hydroxyhexanal, alkenals, e.g., 2-, 3- and 4-pentenal, alkyl 5-formylvalerate, 2-methyl-1-nonanal, 2-methyl 1-decanal, 3-propyl-1-undecanal, pentadecanal, 3-propyl-1-hexadecanal, eicosanal, 2-methyl-1-tricosanal, pentacosanal, 2-methyl-1-tetracosanal, nonacosanal, 2-methyl-1-octacosanal, hentriacontanal, and 2-methyl-1-triacontanal, and the like.

In some embodiments where propylene is the olefin that undergoes the hydroformylation reaction, the product is a mixture of n-butyraldehyde and 2-methylpropionaldehyde. As previously noted, the ratio of linear (N) to branched (I) isomers such as the n-butyraldehyde to 2-methylpropionaldehyde (isobutyraldehyde) ratio is conventionally described as the N:I ratio or N:I.

In one embodiment, the catalyst comprises rhodium, less than one equivalent of polyphosphine, polyphosphine degradation products, and triphenylphosphine (e.g., 30 to 400 moles triarylphosphine to moles rhodium) such that the total polyorganophosphorous compound to rhodium mole ratio is greater than 1.25:1. For example, a process utilizing a catalyst comprised of rhodium and polyphosphine and polyphosphine degradation products may choose to add triphenylphosphine to further lower polyphosphine ligand usage and catalyst deactivation.

Some embodiments of the invention will now be described in more detail in the following Examples. Comparative Experiments are not embodiments of the invention.

### General Procedure

A liquid recycle reactor system is employed that consists of two 1-liter stainless steel stirred tank reactors connected in series. Each reactor is equipped with a vertically mounted agitator and a circular tubular sparger located near the bottom of the reactor. Each sparger contains a plurality of holes of sufficient size to provide the desired gas flow into the liquid body in the reactor. The spargers are used for feeding the olefin and/or syngas to the reactor and can also be used to recycle unreacted gases to each reactor. Each reactor has a silicone oil shell as a means of controlling reactor temperature. Reactors 1 and 2 are further connected via lines to transfer any unreacted gases and lines to allow a portion of the liquid solution containing aldehyde product and catalyst to be pumped from reactor 1 to reactor 2. Hence, the unreacted olefin of reactor 1 is further hydroformylated in reactor 2. Each reactor also contains a pneumatic liquid level controller for maintaining the desired liquid level. Reactor 2 has a blow-off vent for removal of unreacted gases.

A portion of the liquid reaction solution is continuously pumped from Reactor 2 to a catalyst separation zone comprising at least one vaporizer, which consist of a heated vessel at reduced pressure.

The vaporized aldehyde product is condensed and collected in a product receiver; the liquid effluent flows to a vessel (separator) to allow further disengagement of volatile and non-volatile components. A pneumatic liquid level controller controls the level in the separator of non-volatile components, including catalyst solution to be recycled into Reactor 1.

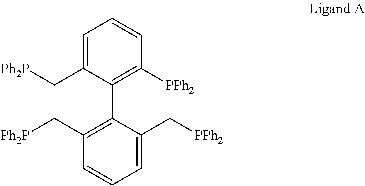

The reactor system is charged with a catalyst solution comprising: (a) rhodium dicarbonyl acetylacetonate (280 ppm rhodium), (b) Ligand A (0.30-0.68 wt %), (c) triphenyl phosphine (6-12 wt. %), (d) solvent mixture comprising 15% by weight of UCAR FILMER IBT (2,2,4-trimethyl-1,3-pentanediol monoisobutyrate, a surrogate for heavies formed by the trimerization of isobutyraldehyde), available from The Dow Chemical Company. and about 85% butyraldehyde, and (e) hydrogen and carbon monoxide to achieve a partial pressure of 10-25 psi. The reactors are then heated to 90° C. under flowing carbon monoxide and hydrogen. Reactor 1 and 2 pressures are maintained at 232 and 142 psig respectively. A propylene olefin stream (consisting of approximately 99.5% propylene) is fed to Reactor 1 at a rate of 2.7-3.5 gram moles per liter of reactor volume per hour. The first vaporizer is operated at 100 psig and 120 to 140° C. and the second vaporizer is operated at 10 psig and 130 to 140° C. (depending on the concentration of heavies to be removed).

The system is operated in a continuous mode for a period of time while monitoring the concentrations of Ligand A, polydentate ligand degradation products, triphenyl phosphine, rhodium, and product aldehydes consisting of N-butyraldehyde (N-Bal) and iso-butyraldehyde (I-Bal). Solutions of Ligand A and/or triphenylphosphine (dissolved in toluene) are added if desired to replenish the levels of these ligands as they are consumed. Occasionally rhodium catalyst precursor is also added if needed. Under normal operating conditions the feed rate of Ligand A is adjusted to maintain target ligand concentrations as described herein. In the following tables, ligand usage rate is g/L/day.

Comparative Experiment A and Example 1 are conducted as described in the General Procedure, with the exception of the catalyst solution being obtained from an operating TPP hydroformylation system wherein the solvent is comprised of mixed butyraldehydes and naturally formed heavies (instead of UCAR FILMER IBT). The initial TPP concentration is 12% to which 0.30 wt. % Ligand A is added. The performance after an initial “break in” period is given below.

| | | | Equiv. total | | |
|---|---|---|---|---|---|
| | | | polyphosphorous | | Ligand A |
| | Days on- | Equiv. Ligand | compounds/Equiv. | | Usage Rate |
| | line | A/Equiv. Rh | Rh | N/I | (g/L/day) |
| Comparative Ex. A | 120-135 | 1.10 | 3.00 | 26 | 0.034 |
| Example 1 | 150-175 | 0.95 | 2.50 | 28 | 0.022 |

Example 1 provides comparable N/I to Comparative Experiment A and does so with lower Ligand A usage.

Comparative Experiments B-D are performed as described in the General Procedure. The results are shown below:

| | | | Equiv. total | |
|---|---|---|---|---|
| | | | polyphosphorous | |
| | Days on- | Equiv. Ligand | compounds/Equiv. | |
| | line | A/Equiv. Rh | Rh | N/I |
| Comparative Ex. B | 100-150 | 1.05 | 2.75 | 28 |
| Comparative Ex. C | 190-210 | 1.10 | 3.30 | 26 |
| Comparative Ex. D | 225-240 | 0.15 | 0.90-1.20 | 8-16 |

Comparative Experiment D demonstrates that allowing the concentration of total polyphosphorous compounds to decline below 1.25 equivalents relative to rhodium and concentration of Ligand A to less than 0.25 equivalents relative to rhodium does not provide the desired performance (i.e., N/I falling to below 10).

Comparative Experiment E is performed according to the General Procedure.

Example 2: Comparative Experiment E transitions into Example 2 by lowering the Ligand A feed rate. The results are shown below:

| | | | Equiv. total | | |
|---|---|---|---|---|---|
| | | | polyphosphorous | | Ligand A |
| | Days on- | Equiv. Ligand | compounds/Equiv. | | Usage Rate |
| | line | A/Equiv. Rh | Rh | N/I | (g/L/day) |
| Comparative Ex. E | 150-300 | 1.20 | 3.60 | 27 | 0.036 |
| Example 2 | 540-560 | 0.95 | 3.75 | 30 | 0.021 |

Example 2 where the Ligand A to rhodium molar ratio is less than 1:1 still provides the desired performance (i.e., N/I is comparable to Comparative Experiment E) but with a 40% reduction in the polyphosphine ligand usage rate.

Comparative Experiments F and G, and Examples 3 and 4 are performed according to the General Procedure with the exception of no TPP being added. The results are shown below:

| | | | Equiv. total | |
|---|---|---|---|---|
| | | | polyphosphorous | |
| | Days on- | Equiv. Ligand | compounds/Equiv. | |
| | line | A/Equiv. Rh | Rh | N/I |
| Comparative Ex. F | 100-115 | 1.25 | 2.75 | 23 |
| Example 3 | 120-132 | 0.75 | 1.75 | 25 |
| Comparative Ex. G | 142-160 | 1.30 | 3.20 | 29 |
| Example 4 | 176-182 | 0.75 | 1.60 | 28 |

Examples 3 and 4 show that the composition of the invention gives good hydroformylation performance with the polyphosphine ligand:transition metal molar ratio below 1:1 and the total polyphosphorous compound to rhodium ratio above 1.25.

Comparative Experiment H and Example 5 are performed according to the General Procedure.

| | | | Equiv. total | | |
|---|---|---|---|---|---|
| | | | polyphosphorous | | Ligand A |
| | Days on- | Equiv. Ligand | compounds/Equiv. | | Usage Rate |
| | line | A/Equiv. Rh | Rh | N/I | (g/L/day) |
| Comparative Ex. H | 142-150 | 1.15 | 2.32 | 19 | 0.092 |
| Example 5 | 153-160 | 0.81 | 1.68 | 22 | 0.044 |

Example 5 shows that reducing the Ligand A feed rate following Comparative Experiment H results in a Ligand A to rhodium ratio less than 1:1, but still provides excellent performance and a lower polyphosphine ligand usage rate.

1. A method for reducing or slowing polyphosphine ligand usage in a continuous hydroformylation process comprising:
(a) contacting an olefin with carbon monoxide, hydrogen and a catalyst, the catalyst comprising (A) a transition metal; (B) a polyphosphine ligand having the following structure: 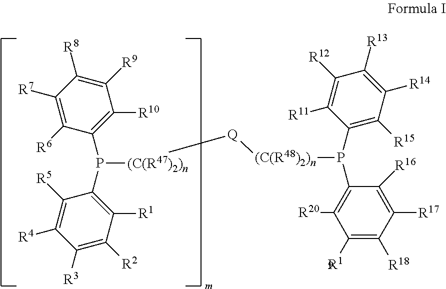
wherein each P is a phosphorous atom, and each of R¹-R²⁰ are independently hydrogen, a C1 to C8 alkyl group, an aryl group, an alkaryl group, or a halogen, R⁴⁷ and R⁴⁸ are independently hydrogen, a C1 to C8 alkyl group, or a C1 to C8 substituted alkyl group, n is 0 or 1, m is 2 or higher, and Q is a m+1 valent organic radical, wherein R⁵ and R⁶ and/or R¹⁵ and R¹⁶ may be links to form bridged structures; and
(C) polydentate ligand degradation products derived from the polyphosphine ligand of Formula I; and
(D) optionally a triarylphosphine having the following structure: 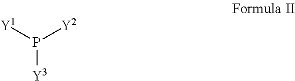
wherein P is a phosphorous atom, and each of Y¹-Y³ are independently an aryl group or substituted aryl group; and
the contacting is conducted in one or more reaction zones and at hydroformylation conditions; and
wherein the ratio of total moles of polyphosphine ligand(s) and polydentate degradation products of the polyphosphine ligands, to the moles of transition metal, is greater than 1.25:1 and
(b) monitoring the levels of polyphosphine ligand(s) and polydentate degradation products of the polyphosphine ligands; and
(c) reducing the feed rate of the polyphosphine ligand(s) such that the total polyphosphorous compounds relative to the moles of transition metal is greater than 1.25:1 and the polyphosphine to transition metal ratio is less than 1:1.

2. The method of claim 1 wherein the polyphosphine ligand to transition metal ratio is greater than 0.25:1.

3. The method of claim 1 wherein the total polyphosphorous compounds relative to the moles of transition metal is greater than 1.5:1

4. The method of claim 1 wherein the amount of triarylphosphine is between 30 to 400 moles relative to the moles of transition metal.

5. The method of claim 1 wherein the polyphosphine ligand is as shown in Formula III. 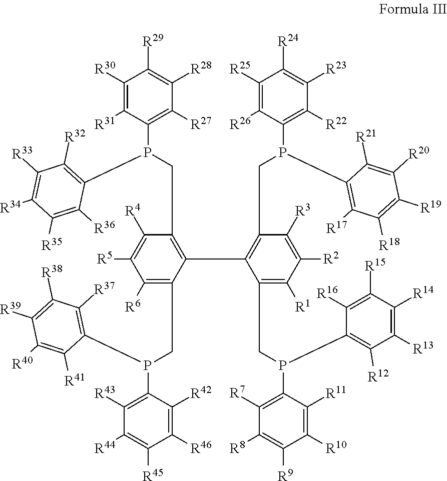
wherein each P is a phosphorous atom, and each of R¹-R⁴⁶ are independently hydrogen, a C1 to C8 alkyl group, an aryl group, an alkaryl group, or a halogen.

6. The method of claim 1 to wherein the triarylphosphine is triphenylphosphine.

7. The method of claim 1 wherein the triarylphosphine is triphenylphosphine and the polyphosphine ligand is Formula IV

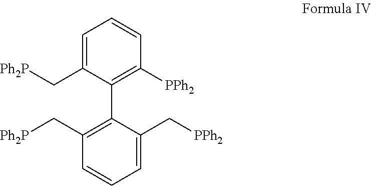

8. The method of claim 1 wherein the mixed aldehydes in the crude product produced in the method has an N/I ratio of above 15.
